SEQUENCE LISTING (11 sequences)

SEQ ID NO: 1 - DNA, 21 bp, artificial
  human PTPH1 siRNA
ccaaaaagucgguaaauaatt

SEQ ID NO: 2 - DNA, 21 bp, artificial
  human PTPH1 siRNA 2
gcaguuaaaaggagguuuctt

SEQ ID NO: 3 - DNA, 21 bp, artificial
  sequence coding for murine PTPH1 siRNA
acctttaaagttaacaaacaa

SEQ ID NO: 4 - DNA, 21 bp, artificial
  sequence coding for murine PTPH1 siRNA
caggagcaaaccaggcatcta

SEQ ID NO: 5 - protein, 27 aa, HIV
GALFLGFLGAAGSTMGAWSQPKKKRKV

SEQ ID NO: 6 - DNA, 20 bp, artificial
  TACE sense primer
gactctagggttctagccca

SEQ ID NO: 7 - DNA, 20 bp, artificial
  TACE antisense primer
cctctgcccatgtatctgta

SEQ ID NO: 8 - DNA, 23 bp, artificial
  murine PTPH1 forward primer
cgtgtcccgagaaatgctagtta

SEQ ID NO: 9 - DNA, 24 bp, artificial
  murine PTPH1 reverse primer
gagatgggtcactgtgtgttcttc

SEQ ID NO: 10 - DNA, 21 bp, artificial
  antisense PTPH1 siRNA
uuauuuaccgacuuuuuggtg

SEQ ID NO: 11 - DNA, 21 bp, artificial
  antisense PTPH1 siRNA
gaaaccuccuuuuaacugctt

(19) United States
(12) Patent Application Publication
(10) Pub. No.: US 2011/0015254 A1
(43) Pub. Date: Jan. 20, 2011

(54) PTPH1 Inhibitors for the Treatment of Alzheimer's Disease

(75) Inventors: Claudia Patrignani, Cattolica (IT); Valeria Muzio, Banchette (IT); Maria-Chiara Magnone, Basel (CH); Paola Zaratin, Milan (IT); Beatrice Greco, Ivrea (IT)

Correspondence Address: SALIWANCHIK LLOYD & SALIWANCHIK;A PROFESSIONAL ASSOCIATION, PO Box 142950, GAINESVILLE, FL, 32614, US

(73) Assignee: MERCK SERONO SA, COINSINS, VAUD (CH)

(21) Appl. No.: 12/934,195

(22) PCT Filed: Mar. 17, 2009
(86) PCT No.: PCT/EP2009/053151

§ 371 Date: Sep. 23, 2010

Related U.S. Application Data
(60) Provisional application No. 61/072,037, filed on Mar. 27, 2008.

(30) Foreign Application Priority Data
Mar. 25, 2008 (EP) 08102890.4

(51) Int. Cl. A61K 31/7088; A61P 25/28; G01N 33/566

(52) U.S. Cl. 514/44 A; 435/7.21; 435/7.1

(57) ABSTRACT

The present invention relates to the use of PTPH1 inhibitors in the prevention or treatment of Alzheimer's Disease, or a symptom thereof. The present invention also relates to a method of identifying compounds useful in the prevention or treatment of Alzheimer's Disease, or a symptom thereof.

### FIELD OF THE INVENTION

The invention concerns treatment of neurological diseases, in particular of Alzheimer's Disease. It relates to a PTPH1 inhibitor for the prevention or treatment of Alzheimer's Disease, or a symptom thereof. It also relates to methods of identifying PTPH1 inhibitors that are useful in the prevention or treatment of Alzheimer's Disease, or symptoms thereof.

### BACKGROUND OF THE INVENTION

In Alzheimer's disease, the ability to remember, think, understand, communicate, and control behavior progressively declines because brain tissue degenerates. This disease accounts for most dementias in older people, in particular aged above 60.

Diagnosis is generally based on anamnesis, physical examination and the results of tests, such as mental status tests, blood and urine tests, and computed tomography (CT) or magnetic resonance imaging (MRI). Based on the information obtained, other types and causes of dementia can generally be excluded. Patients suffering from Alzheimer's disease also generally have a low level of acetylcholine in the brain.

Presently, treatment of Alzheimer's disease is the same as that of other dementias. Cholinesterase inhibitors may stabilize or slightly improve mental function (including memory).

Alzheimer's Disease is a neurodegenerative disorder that is characterized by a progressive cognitive impairment, personality changes and specific neuropathological abnormalities.

The brain areas typically involved in Alzheimer's Disease are the entorhinal cortex, hippocampus, parahippocampus gyrus, amygdala, frontal, temporal parietal and occipital association cortices. Many neurons in these brain regions contain large non membrane-bound bundles of abnormal fibers, which occupy much of the perinuclear cytoplasm: the neurofibrillary tangles, composed of hyperphosphorylated tau filaments (Selkoe et al. 2001). Neurofibrillary tangles together with amyloid beta depositions lead to massive neuronal degeneration, to brain atrophy and to the subsequent cognitive and memory impairment, features of Alzheimer's Disease.

The earliest changes in Alzheimer's Disease brains occur in the anterior medial temporal lobe, which includes hippocampus and entorhinal cortex (Devanand et al., 2007). The entorhinal cortex is a memory center. It receives inputs from cortical areas, as the prefrontal cortex, and projects to the hippocampus, mainly to CA1 and dentate gyrus areas. The entorhinal cortex system plays an important role in memory consolidation, memory optimization and sleep.

The atrophy of hippocampus, neocortex and entorhinal cortex detected in Alzheimer's Disease patients leads to a malfunction of the memory and cognitive circuits.

The importance of an early diagnosis has led to a growing number of MRI studies on mild cognitive impairment (MCI), considered as an initiation phase to Alzheimer's Disease (Chetelat et al 2002; Karas et al 2004).

Whitwell and colleagues following the progression of the cognitive impairment from MCI to Alzheimer's Disease by MRI have confirmed the early atrophy of several key brain areas such as the left amygdala, bilateral hippocampus, entorhinal cortex and fusiform gyrus. By the time the subjects had progressed to a clinical diagnosis of Alzheimer's Disease the pattern of cerebral atrophy detected on MRI had become dramatically more widespread with more severe involvement of the medial temporal lobes and the tempo-parietal association cortices and substantial involvement of the frontal lobes. These regions are all typically involved in Alzheimer's Disease (Fox et al., 1996; Jack et al., 2004; Frisoni et al., 2002) and these widespread patterns of loss likely correspond to the worsening cognitive functioning that led to the progression to Alzheimer's Disease.

Typically neurofibrillary tangles occur first in the entorhinal cortex and the hippocampus (transentorhinal stages I-II of Alzheimer's Disease), before spreading out into the amygdale, the basolateral temporal lobe (limbic stages III-IV) and then into the isocortical association areas (isocortical stages V-VI). A pathological diagnosis of high-probability AD is given when isocortical areas are involved. The patterns of atrophy observed at each disease stage are usually bilateral, although showing greater involvement of the left hemisphere (Boxer et al., 2003; Karas et al., 2003).

The hippocampus is strongly involved not only in the early phases, but also during progression of the disease. Several studies showed progressive atrophy throughout the disease course, with the severity of hippocampal loss detected at MRI, increasing from MCI to early AD (Whitwell et al., 2007). The gray matter loss detected on MRI is predominantly located in the anterior regions of the hippocampus in MCI patients, and then progresses to involve the posterior hippocampus in early AD (Whitwell et al., 2007). Several studies suggest that the anterior portion of the hippocampus is more susceptible to degenerative change than the posterior portion.

On a molecular level, the most common feature of Alzheimer's Disease is the progressive deposition of the Aβ peptides in senile plaques. The plaques are composed of extracellular deposits of a heterogeneous mixture of Aβ peptides (40-42/43 amino acids in length), which are derived from the enzymatic cleavage of the amyloid precursor protein (APP). In normal healthy individuals, Aβ peptides are present only in small quantities as soluble monomers that circulate in cerebrospinal fluid and blood (Parvathy et al., 1999). In Alzheimer's Disease patients, on the contrary, their levels are significantly increased, thus leading to Aβ accumulation as insoluble, fibrillar plaques. This observation led to the formulation of the “APP hypothesis”. APP (amyloid precursor protein) is a transmembrane protein normally expressed in the brain that can be processed by 2 different pathways. The amyloidogenic pathway consists in the cleavage of APP between residues Met⁶⁷¹ and Asp⁶⁷² by a β-secretase, yielding to sAPPβ and C99 fragments. C99 fragments are processed by a γ-secretase and further cut into amyloid β peptides (Aβ).

Aβ accumulates in neurons and forms insoluble aggregates, so called senile plaques that represent the major hallmark of Alzheimer's Disease.

APP can also be processed by α-secretases that cleave the protein within the Aβ domain between Lys⁶⁸⁷ and Leu⁶⁸⁸, thus producing a large soluble αAPP domain and a C-terminal fragment containing P3 and C83. αAPP fragments are then cleaved by γ-secretase at residue 711 or 713 with the following release of P3 fragment. This last pathway, called non-amyloidogenic, does not yield Aβ peptides. Hence, shunting APP towards the α-secretase pathway may be beneficial in lowering Aβ peptide levels.

In fact, most of the recent studies on new therapies for Alzheimer's Disease are focused on the production of α-secretase enhancers (Citron et al., 2004).

TNF Alpha Convertase Enzyme (TACE) is one of the most important α-secretases. It belongs to the ADAM family protein (A Disintegrin And Metalloproteinase) and besides its role as an α-secretase, TACE is responsible for the shedding of cytokines and chemokines as TNF-α, TGF-α, L-selectin, p75 and p55, TNF receptors, IL-1R2.

As explained above, enhancing TACE activity might be a way to reduce Aβ plaques deposition. However, since TACE is involved in other crucial pathways for cell survival, the effects of TACE up-regulation in vivo need to be explored. Animal models for TACE over-expression can be obtained either by creating transgenic mice for TACE or by knocking out genes encoding TACE inhibitors.

The role of TACE in Multiple Sclerosis pathogenesis and in Experimental Autoimmune Encephalomyelitis (EAE) models has been also investigated. Recently it has been shown that increased expression of TACE in peripheral blood mononuclear cells (PBMC) derived from Multiple Sclerosis (MS) patients appears to precede blood brain barrier leakage and is also observed in T infiltrating cells in active and chronic MS plaques (Seifert et al., 2002). It is, furthermore, differentially regulated in MS subforms suggesting that different regulatory mechanisms of TACE-TNFα release may be involved in the different clinical subtypes of MS (Comabella et al., 2006).

In EAE models, increased TACE expression has been described in astrocytes and invading macrophages in the spinal cords of rat acute EAE at the peak of the disease. Similarly increased TACE expression in the spinal cord of relapsing-remitting EAE in mice has been reported during the primary inflammatory phase. However, no information is available on TACE regulation in these pathologies (Plumb et al., 2005; Toft-Hansen et al., 2004).

PTPH1 is a non-transmembrane protein tyrosine phosphatase that was shown to be expressed in the thalamic areas connected to the cortex. PTPH1 expression profile in rat brain is localized in most thalamic nuclei, hippocampus, cerebellum, entorhinal cortex and cortex (Sahin et al., 1995). PTPH1 has been recently shown to interact with TACE in vitro. In particular, PTPH1 seems to down-regulate TACE in vitro by binding to its PDZ domain (Zheng et al., 2002).

So far, there has been no indication in the prior art that PTPH1 inhibitors could be beneficial in treatment or prevention of Alzheimer's Disease.

### SUMMARY OF THE INVENTION

In a first aspect, the invention relates to an inhibitor of PTPH1 for preventing or treating Alzheimer's Disease, or a symptom thereof.

In a second aspect, the invention relates to a method of identifying a compound useful in preventing or treating Alzheimer's Disease comprising:
contacting PTPH1 in the presence or absence of a candidate compound in vitro; and
comparing the activity of PTPH1 in the presence of the candidate compound to the activity of PTPH1 in the absence of the candidate compound,
   wherein a compound decreasing the activity of PTPH1 is identified as a compound useful in preventing or treating Alzheimer's Disease, or a symptom thereof.

### BRIEF DESCRIPTION OF THE DRAWINGS

FIG. 1a-b) Blot analysis of pro-TACE and TACE in cerebellum and c-d) in hippocampus; e-f) percentage of activated TACE form present in control and diseased conditions. T-test performed: p<0.05; *: p<0.01; ***: p<0.001; CTRL WT/KO: PTPH1-WT/KO mice immunized with CFA; EAE-WT/KO: PTPH1-WT/KO mice immunized with CFA and MOG peptide.

FIG. 2: a-b) Blot analysis of pro-TACE and TACE in striatum and c-d) in cortex; e-f) percentage of activated TACE form present in control and diseased conditions. T-test performed: p<0.05; *: p<0.01; ***: p<0.001; CTRL WT/KO: PTPH1-WT/KO mice immunized with CFA; EAE-WT/KO: PTPH1-WT/KO mice immunized with CFA and MOG peptide.

FIG. 3: a-b) Blot analysis of pro-TACE and TACE in midbrain and c-d) in pontine region; e-f) percentage of activated TACE form present in control and diseased conditions. T-test performed: *p<0.05; **: p<0.01; ***: p<0.001; CTRL WT/KO: PTPH1-WT/KO mice immunized with CFA; EAE-WT/KO: PTPH1-WT/KO mice immunized with CFA and MOG peptide.

FIG. 4: a-e) TACE activity measured in different brain areas by a fluorometric kit; CTRL WT/KO: PTPH1-WT/KO mice immunized with CFA; EAE-WT/KO: PTPH1-WT/KO mice immunized with CFA and MOG peptide.

### DETAILED DESCRIPTION OF THE INVENTION

The present invention is based on the finding that in a mouse lacking the PDZ and catalytic domains of PTPH1, in which CNS inflammation had been induced, increased TACE expression and activity occurred in those brain regions, which are particularly involved in the development and progression of Alzheimer's Disease (Alzheimer's Disease). As explained above in the Background of the Invention, TACE has an α-secretase activity and cleaves APP (amyloid precursor protein) in such a way as to generate non-pathological αAPP fragments. The enhanced TACE expression and activity was particularly pronounced in the hippocampus, which is the key brain area that undergoes atrophy both in the initiation phase of AD as well as in disease progression.

Therefore, the invention relates to an inhibitor of PTPH1 for prevention or treatment of AD. The invention also relates to the use of a PTPH1 inhibitor for the preparation of a medicament for prevention or treatment of AD, or a symptom thereof.

The term “prevention” within the context of this invention refers not only to a complete prevention of a certain symptom of AD, but also to any partial or substantial prevention, attenuation, reduction, decrease, diminishing or alleviating of any symptom or consequence of AD before or at early onset of disease.

Prevention of AD can e.g. be foreseen in individuals displaying one or more risk factors of AD. The best-studied “risk” gene is the one that encodes apolipoprotein E (apoE). The apoE gene has three different forms (alleles), namely apoE2, apoE3, and apoE4. The apoE4 form of the gene has been associated with increased risk of AD in most populations studied. The frequency of the apoE4 version of the gene in the general population varies, but is always less than 30% and frequently 8%-15%. Persons with one copy of the E4 gene usually have about a two to three fold increased risk of developing this disease. Persons with two copies of the E4 gene (usually around 1% of the population) have about a nine-fold increase in risk. At least one copy of the E4 gene is found in 40% of patients with sporadic or late-onset AD. Those individuals are a preferred group to be treated with the PTPH1 inhibitor, in line with the present invention.

The term “treatment” within the context of this invention refers to any beneficial effect on progression of disease, including attenuation, reduction, decrease, diminishing or alleviation of the pathological development or one or more symptoms developed by an AD patient during the disease, including the slowing-down of the progress of the disease, or improvement of any symptom thereof.

As explained in the Background, pathological symptoms of AD are the neurofibrillary tangles as well as the progressive deposition of Aβ peptides in so-called senile plaques.

Therefore, in an embodiment of the invention, said symptom of AD to be treated or prevented in accordance with the present invention is selected from the group consisting of the presence of neurofibrillary tangles, Aβ depositions, neuronal degeneration, and brain atrophy.

Clinical symptoms of Alzheimer's Disease include e.g. dementia, impairment of memory, in particular short-term memory, personality changes, apathy, agitation, irritability, confusion or disorientation. The symptoms further include psychiatric symptoms relating e.g. to depression, hallucinations, anxiety, and sleep disorders related to AD.

Therefore, in a preferred embodiment, the inhibitor is for prevention or treatment of a symptom of Alzheimer's Disease selected from dementia, impairment of memory (in particular short-term memory), personality changes, apathy, agitation, irritability, confusion or disorientation, depression, hallucinations, anxiety, and sleep disorders. It is understood that in the context of the present invention, those symptoms are related to AD and not to any other neurological disease or disorder.

AD is characterized by atrophy of the hippocampus, neocortex and entohinal cortex. In particular, the disease progressed from the early atrophy of left amygdale, hippocampus, entorhinal cortex and fusiform gyrus to the involvement of medial temporal lobes and tempo-parietal association cortices and frontal lobes.

In accordance with the present invention, any one of the defined stages of Alzheimer's Disease can be treated or prevented using a PTPH1 inhibitor, i.e. stages I-II (transentorhinal stage), II-IV (limbic stage) or V-VI (isocortical stage), as determined e.g. by MRI (Magnetic Resonance Imaging) (explained e.g. in Thompson and Toga, 2008) or by PET (positron emission tomography (Scarmeas et al., 2004).

In one embodiment, the invention relates to a PTPH1 inhibitor for treating or preventing early stage Alzheimer's Disease, preferably transentorhinal stage I-II, as determined e.g. by MRI.

In an embodiment of the invention, the inhibitor of PTPH1 decreases the enzymatic activity of PTPH1. Such an inhibitor can e.g. be a small molecular weight compound. The enzymatic activity of PTPH1 can be measured e.g. in an assay as described in Example 3 below (the so-called DiFMUP assay), by measuring the extent of dephosphorylation of an adequate substrate or the extent of free phosphate generated by the PTPH1 activity.

Such an assay can be used to determine the IC₅₀ of any PTPH1 inhibitor. In an embodiment, the PTPH1 inhibitor has an IC₅₀ for PTPH1 being lower than 6 μM or lower than 5 μM or lower than 4 μM or lower than 3 μM or lower than 2 μM.

In a further embodiment, the inhibitor of PTPH1 decreases PTPH1 expression.

PTPH1 expression can be measured e.g. in a cell expressing PTPH1 by comparing the level or activity of PTPH1 in the absence or presence of the inhibitor.

In accordance with the present invention, PTPH1 activity or expression can be measured in an assay as described in Example 2. A cell line such as e.g. a CHO and/or HEK293 cell line are transfected to express or overexpress APP. For instance, the Swedish variant of human APP having the two amino acid substitutions Lys670Asn (K670N) and Met671Leu (M671L) is suitable as it leads to high secretion of APP into the medium. The cells are being incubated with a PTPH1 inhibitor, such as a small molecular weight compound, or transfected with an siRNA specific for PTPH1. The extent of PTPH1 activity can be measured in the DiFMUP assay. The extent of pathogenic APP peptides, such as e.g. the Aβ40 peptide, in can be measured in the cell extract or supernatant, in an ELISA type assay, for instance. The PTPH1 inhibitor reduces the amount of pathogenic APP peptides such as the Aβ40 peptide and is thus suitable for prevention or treatment of Alzheimer's Disease.

In an embodiment of the invention, the PTPH1 inhibitor is a siRNA specific for PTPH1, preferably human PTPH1. siRNA are generally approximately 19-23 base pairs in length and contain two nucleotide 3′ overhangs.

A siRNA of the invention can e.g. have a sequence of SEQ ID NO: 1 (CCAAAAAGUCGGUAAAUAAtt) or SEQ ID NO: 2 (GCAGUUAAAAGGAGGUUUCtt).

In an embodiment, the siRNA is chemically stabilized and cholesterol-conjugated siRNA as described e.g. by Soutschek et al., 2004. Such stabilized and conjugated siRNAs have improved pharmacological properties in vitro and in vivo. Chemically stabilized siRNAs with partial phosphorothioate backbone and 2′-O-methyl sugar modifications on the sense and antisense strands show enhanced resistance towards degradation by exo- and endonucleases in serum and in tissue homogenates. The conjugation of cholesterol to the 3′ end of the sense strand of a siRNA molecule, e.g. by means of a pyrrolidine linker (thereby generating chol-siRNA), further improves pharmacological half-live of siRNAs and leads to penetration of the siRNA into the cytosol, presumably by using the LDL (low density lipoprotein) receptor transporter system.

Further delivery systems of small interfering RNA (siRNA) have been described. For instance, as described by Sato et al., 2007, cationic comb-type copolymers (CCCs) possessing a polycationic backbone (less than 30 weight (wt) %) and abundant water-soluble side chains (more than 70 wt. %) as a siRNA carrier lead to prolonged blood circulation time. The CCC and siRNA can also be separately administered, e.g. at 20 min interval, with blood circulation of post-injected siRNA still being significantly increased.

Also, chemical modifications like 2′-O-methyl ribonucleotides and phosphorothioate linkages in the backbone confer resistance to nuclease attack, while enlarging the molecules to about 50 kD, can prevent loss through kidney filtration.

Another possibility is to package siRNAs inside liposomes, which protect the siRNA from degradation and kidney clearance. Linkage of siRNA to peptides or single chain antibodies have been described as suitable delivery systems for siRNAs as well.

siRNAs can not only be exogenously administered as synthetic chemicals complexed with or covalently attached to a non-viral delivery system. They can also be produced intracellularly from short hairpin RNA (shRNA) constructs, that are normally introduced into cells by the use of viral vectors.

The use of peptide transduction domains or cell penetrating peptides for exogenous siRNA delivery is known as well (reviewed by Meade and Dowdy, 2008). Peptide transduction domains (PTD), also called cell penetrating peptides (CPPs) are a class of small cationic peptides of approximately 10-30 amino acids in length that have been shown to engage the anionic cell surface through electrostatic interactions and rapidly induce their own cellular internalization through various forms of endocytosis. After being internalized within endocytic bodies, PTD are capable of endocytic vesicle escape and gain access to the intracellular environment. Some of the most well characterized PTD thus far are TAT peptide, penetratin, transportan, poly-arginine and MPG. These cationic peptides have also been shown to enhance the cellular uptake of covalently coupled cargo, making them attractive candidates for applications where the intracellular delivery of large macromolecules is desirable.

For instance, one peptide enhancing cellular uptake of siRNAs is called MPG. MPG is a 27 amino acid amphipathic peptide composed of a basic domain from the nuclear localization signal (NLS) of SV40 large T antigen and a hydrophobic domain derived from HIV-1 gp41 (GALFLGFLGAAGSTMGAWSQPKKKRKV—SEQ ID NO: 5).

Polyarginine peptides of 8 to 10 amino acids are used as well for enhanced transfer of siRNAs over the cell membrane.

Entrapment of siRNAs in endosomal vesicles can be circumvented by a designed endosomolytic EB1 peptide. EB1 peptide is a modified penetratin peptide that has specifically placed histidine insertions that theoretically induce an alpha helical formation upon protonation in the acidic endosome environment. This conformation change can lead to endosomal disruption and consequently, enhanced endosomal release of functional siRNA cargo.

Strategies for targeted gene silencing by siRNA in the central nervous system are known as well (reviewed by Pardridge, 2007). For RNA interference of the brain, the nucleic acid-based drug must first cross the brain capillary endothelial wall, which forms the blood-brain barrier (BBB) in vivo, and then traverses the brain cell plasma membrane. Plasmid DNA encoding for short hairpin RNA (shRNA) may be delivered to the brain following intravenous administration with pegylated immunoliposomes (PILs). The plasmid DNA is encapsulated in a 100 nm liposome, which is pegylated, and conjugated with receptor specific targeting monoclonal antibodies. SiRNA duplexes can be delivered with the combined use of targeting MAb's and avidin-biotin technology. The siRNA is mono-biotinylated in parallel with the production of a conjugate of the targeting monoclonal antibody and streptavidin.

In an embodiment of the present invention, the PTPH1 inhibitor is administered or prepared, formulated or adapted for administration in combination with an anti-Alzheimer's Disease compound selected from cholinesterase inhibitors or a glutamate inhibitor.

The combined treatment can be used for simultaneously, sequentially or separately. The PTPH1 inhibitor and the further compound can be co-administered or adapted or formulated for combined administration.

The cholinesterase inhibitor may e.g. be selected from donepezil hydrochloride, rivastigmine, galantamine or tacrine.

The glutamate inhibitor may e.g. be memantine.

In accordance with the present invention, the PTPH1 inhibitor may also be administered or prepared, formulated or adapted for administration, in combination with antipsychotic agents such as mood-stabilizing anticonvulsants, trazodone, anxiolytics, or beta-blockers.

The invention further relates to a method of treatment of Alzheimer's Disease comprising administering to an individual or patient in need thereof a therapeutically effective amount of a PTPH1 inhibitor, preferably together with a pharmaceutically acceptable carrier.

A “therapeutically effective amount” is such that when administered, the PTPH1 inhibitor results in inhibition of the biological activity of PTPH1. The dosage administered, as single or multiple doses, to an individual will vary depending upon a variety of factors, including pharmacokinetic properties of the PTPH1 inhibitor, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art, as well as in vitro and in vivo methods of determining the inhibition of PTPH1 in an individual.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, intracranial, epidural, topical, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the PTPH1 can be administered together with other components such as pharmaceutically acceptable surfactants, excipients, carriers, diluents and vehicles.

For parenteral (e.g. intravenous, subcutaneous, intramuscular) administration, the active agent can be formulated as a solution, suspension, emulsion or lyophilized powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The invention further relates to a method of identifying a compound useful in preventing or treating AD, or a symptom thereof, comprising:
contacting PTPH1 in the presence or absence of a candidate organic compound in vitro; and
comparing the activity of PTPH1 in the presence of the candidate organic compound to the activity of PTPH1 in the absence of the candidate organic compound,
   wherein a compound decreasing the activity of PTPH1 is identified as a compound useful in preventing or treating Alzheimer's Disease, or a symptom thereof.

In an embodiment, the candidate compound is tested on a cell stably expressing APP, wherein the activity of PTPH1 is being assessed by measuring the amount of Aβ peptides in the cell extract or supernatant, and wherein a lower amount of Aβ peptides in presence of the candidate compound as compared to the absence of the candidate compound is indicative of the utility of the candidate compound in treating or preventing Alzheimer's Disease, or a symptom thereof.

The candidate compound can e.g. be a small molecular weight inhibitor of PTPH1, or a siRNA inhibiting PTPH1. Suitable siRNAS are e.g. RNAs having the sequence of SEQ ID NO: 1 (CCAAAAAGUCGGUAAAUAAtt), SEQ ID NO: 2 (GCAGUUAAAAGGAGGUUUCtt) or SEQ ID NO: 3 (ACCTTTAAAGTTAACAAACAA).

The cell can e.g. be a CHO or a HEK293 cell. The amount of amyloid 13 peptides can be measured e.g. using an appropriate antibody in an ELISA.

The extend to which amyloid β peptides are diminished by the PTPH1 inhibitor can be at least 10% or 20% or 30% or 40% or 50% lower than in the absence of the inhibitor.

In a preferred embodiment, the compound decreasing the activity of PTPH1 is further tested in an animal model of AD. Such an experimental model, e.g. a mouse model, displays hallmark Alzheimer's Disease pathology signs such as amyloid plaques, neurofibrillary tangles, reactive gliosis, dystrophic neurites, neuron and synapse loss, and brain atrophy and in parallel behaviorally mimic the cognitive decline observed in humans. Magnetic resonance (MR) microscopy (MRM) can detect amyloid plaque load, development of brain atrophy, and acute neurodegeneration. One such mouse model is e.g. the mouse harboring two familial AD-linked genes (human APP Swedish and presenilin1-ΔE9), in which levels of Aβ (especially Aβ₄₂) are elevated, leading to the formation of amyloid plaques (Sheng et al., 2002). Another useful mouse model to study AD pathophysiology is the apoE4 (Δ272-299) transgenic mouse. Human apolipoprotein (apo) E, a 34-kDa protein composed of 299 amino acids, occurs as three major isoforms, apoE2, apoE3, and apoE4 (Mahley et al., 2000). ApoE4 is a major risk factor for AD in humans, and also accelerates the onset of the disease (Corder et al., 1993). It has been shown that apoE undergoes proteolytic cleavage in AD brains and in cultured neuronal cells, leading to the accumulation of carboxyl-terminal-truncated fragments of apoE that are neurotoxic (Huang et al 2001). These transgenic mice expressing the carboxyl-terminal-cleaved product, apoE4 (Δ272-299), at high levels in the brain displayed AD-like neurodegenerative alterations, including hyperphosphorylated tau, resembling neurofibrillary tangles, but they die at 2-3 months of age. Low level apoE4 (Δ272-299) expressing mice survived longer but showed impaired learning and memory at 6-7 months of age (Harris et al., 2003). A more recent mouse model is the THY-Tau22 mouse that expresses human 4-repeat tau mutated at sites G272V and P301S under a Thy1.2-promotor. The pathology in these mice starts in the hippocampus and they display neurofibrillary tangles, PHF, and tau hyperphosphorylation leading to memory deficits (Schindowski et al., 2007)

All of these models are well known to the person skilled in the art and are suitable to further test PTPH1 inhibitors for treatment of AD.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning a range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### Example 1

### Analysis of PTPH1 KO Mice

### Materials and Methods

### Animals

PTPH1 knockout (KO) and wild type (WT) littermates (F2 generation, 87.5% C57Bl/6-12.5% 129S6SvEv, 5 months old) were used for a mouse chronic experimental autoimmune encephalitis (EAE) experiment. Mice were housed at two/three per cage and maintained in a 12:12 hours light:dark cycle (lights on at 7 am) at 21±1° C. with food and water available ad libitum.

### PTPH1 KO Design

PTPH1 KO mice were obtained from Regeneron Inc. (USA) with a proprietary Loss-of-Native-Allele procedure described by Valenzuela et al., (2003). The genomic sequence of PTPH1 from exon19 to exon27 was replaced in frame with PTPH1 initiation codon by a LacZ-Neo cassette. This insertion removed a genomic sequence of approximately 30 KB encoding for the PDZ domain and for the catalytic domain of the protein.

### Experimental Autoimmune Encephalomyelitis (EAE) Immunization Procedure

8 PTPH1-WT and 9 PTPH1-KO female mice littermates (5 month-old) were immunized as follows:

On day 0 immunization were conducted by injecting s.c. in the left flank 0.2 mL of an emulsion composed of 200 μg MOG₃₅₋₅₅ peptide (Neosystem, Strasbourg, France) in Complete Freund's Adjuvant (CFA, Difco, Detroit, U.S.A.) containing 0.5 mg of Mycobacterium tuberculosis. Immediately after, they received an i.p. injection of 500 ng pertussis toxin (List Biological Lab., Campbell, Calif., U.S.A.) dissolved in 400 μL of buffer (0.5 M NaCl, 0.017% Triton X-100, 0.015 M Tris, pH=7.5).

On day 2 the animals were given a second i.p. injection of 500 ng pertussis toxin.

On day 7, the mice received a second dose of 200 μg of MOG₃₅₋₅₅ peptide in CFA injected s.c. in the right flank. Starting approximately from day 8-10, this procedure resulted in a gradually progressing paralysis, arising from the tail and ascending up to the forelimbs.

4 mice per genotype were immunized just with CFA (no MOG peptide) to be used as healthy controls. Clinical score and body weight were recorded daily. Mice were scored as follows: 0, no sign of disease; 0.5, partial tail paralysis; 1, tail paralysis; 2, partial hind limb paralysis; 3, complete hind limb paralysis; 4, hind limb and forelimb paralysis; 5, moribund or dead.

All the mice were sacrificed at 49-50 days post immunization (dpi) by an overdose of intraperitoneal injection of thiopental.

### Western Blot

Brains were freshly removed and microdissected in different areas (olfactory bulbs, cerebellum, hippocampus, striatum, cortex pontine region and midbrain), then snap frozen.

Protein extraction was performed by mechanical homogenation in Cell extraction buffer provided by R&D Systems (α-secretase activity kit #FP001). The method used allowed using the samples for Western blot and for α-secretase activity. Western blot analysis was performed on 30-50 μg of proteins. Lysates were run on an 8% SDS-page and transferred to nitrocellulose membrane (BioRad). Blots were cut at the level of 50 KDa. The blots up to 50 KDa were incubated in rabbit anti-TACE (1:2000, Sigma) overnight at 4° C. with gentle rocking. Following washing, blots were incubated in HRP-linked anti-rabbit IgG (1:1000, Cell Signaling Tech.) for 1 hour, followed by washing and detection by ECL (Pierce). The blots from 50 KDa were probed using a rabbit anti β-actin (1:250, Sigma). The bands have been detected by the ChemiDoc™ XRS system, PC, an imaging system using a supercooled 12-bit CCD camera with 1.3 megapixel resolution (BioRad, #170-8070). The intensity of the bands have been analyzed by the Quantity One® software for PC.

### α-Secretase Activity Test

The same protein extract was tested for TACE activity by using a fluorometric kit of R&D Systems (α-secretase activity kit #FP001). Cleavage of the α-secretase/TACE-specific peptide conjugated to the reporter molecules EDANS and DABCYL is induced by TACE and physically separates the EDANS and DABCYL allowing for the release of a fluorescent signal. The level of α-secretase enzymatic activity in the cell lysate is proportional to the fluorometric reaction. The analysis was run in duplicates and the results were expressed as fold increases in fluorescence over background controls (reactions without cell lysate or substrate).

### Statistics

Clinical score of the chronic EAE mice was expressed as mean±SEM and was analyzed by a one-way ANOVA followed by a Fisher post-hoc test. Data of WB, α-secretase test were analyzed by T-test.

### Results

The role of PTPH1 on TACE was studied in a mouse model of CNS inflammation, namely the MOG induced chronic EAE. In this model, as in AD, an inflammation-driven pathology of the CNS occurs, in which TACE is known to play an important role. This experiment demonstrated that lack of PTPH1 has an effect on TACE activity and expression in the inflamed brain in vivo, in particular in those areas involved in development and progression of Alzheimer's Disease.

### Disease Course

WT mice developed clinical signs of paralysis starting at 15 dpi and reached a chronic and stable disease at 21 dpi with a score value of about 3 (complete hind limb paralysis, not shown). KO-mice started to develop signs of paralysis at 13 dpi. However no significant differences in the onset of the disease, in the severity (clinical score, not shown), and mortality (not shown) were recorded in comparison to WT mice. PTPH1-KO and PTPH1-WT mice receiving CFA (CTRL) did not show any signs of EAE.

### TACE Expression

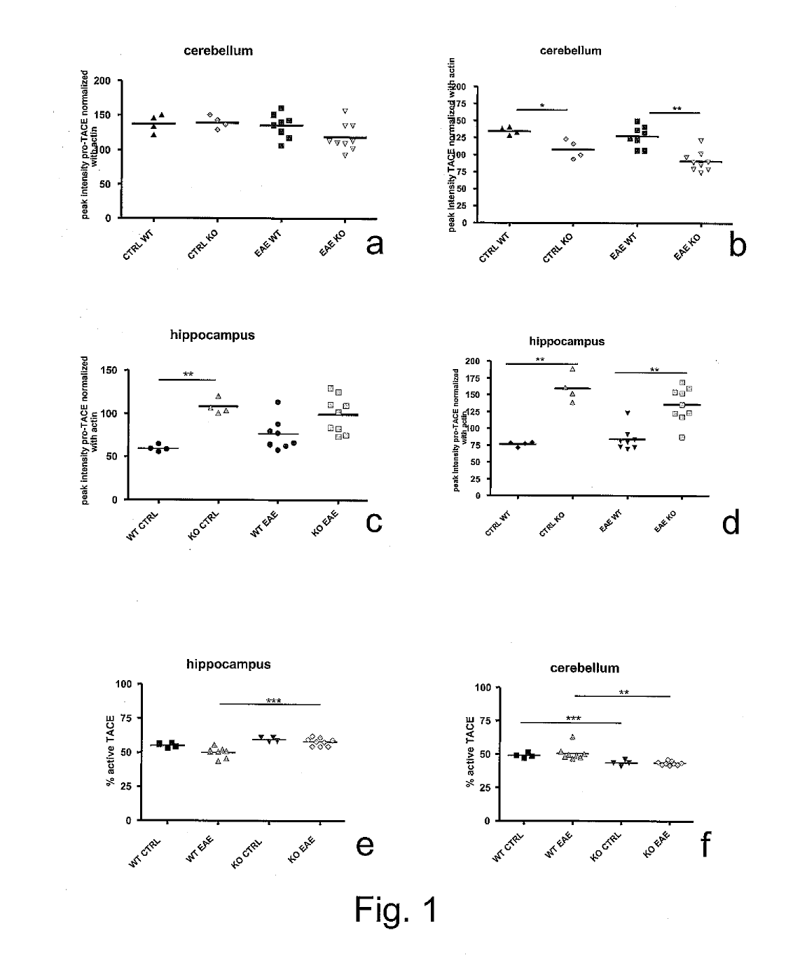

Cerebellum: TACE subforms, pro-TACE, catalytically inactive (130 KDa) and the mature active form (80 KDa) were detected by rabbit anti-TACE. In the cerebellum there was no difference in pro-TACE expression both in normal and in diseased conditions (FIG. 1a). TACE mature form expression seemed to be down-regulated in PTPH1-KO mice, both in control and in diseased conditions (P_{ctrl}=0.0486; P_{EAE}=0.0053 (WT vs. KO)) (FIG. 1b). The percentage of activated TACE is the measure of the quantity of catalytically active protein over the total. The cerebellum of PTPH1-KO mice displayed a significant decrease of percentage of activated TACE in control and diseased conditions compared to Healthy and EAE WT (P_{ctrl}<0001; P_{EAE}<0.01 (WT vs. KO)) (FIG. 1f).

Hippocampus: In this brain region pro-TACE was up-regulated in PTPH1-KO mice compared to the WT littermates in control (P_{ctrl}=0.0025 (WT vs. KO)) and diseased conditions (no sign P_{EAE}=0.0782 (WT vs. KO)) (FIG. 1c); also the mature form was significantly higher in PTPH1-KO hippocampus compared to the WT ones in both conditions (P_{ctrl}=0.0043; P_{EAE}=0.0011 (WT vs KO)) (FIG. 1d). As for the percentage of activated TACE, EAE PTPH1-KO hippocampus showed a significantly higher expression compared to EAE WT littermates (P_{EAE}<0.001 (WT vs. KO)) (FIG. 1e). This trend in TACE expression indicates that this protease is inhibited PTPH1.

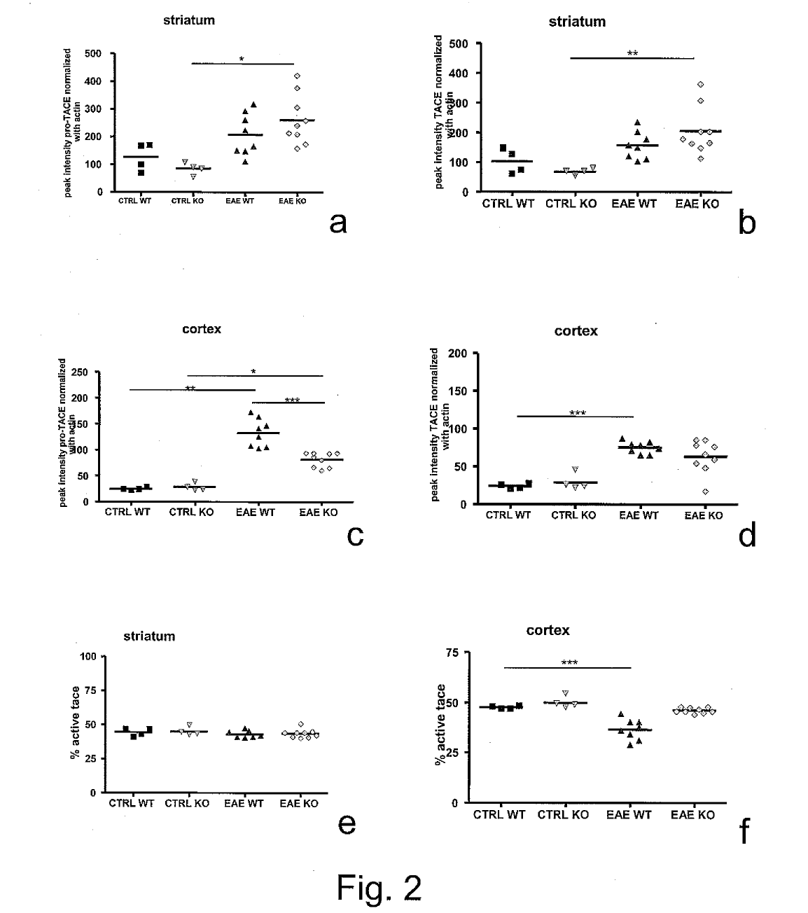

Striatum: A significant increase of both TACE forms has been recorded in PTPH1-KO mice in disease conditions (P_{pro-TACE}=0.0124; P_{TACE}=0.0074 (KO_{EAE} vs. KO_{ctrl})) (FIG. 2a-b) and no differences have been recorded between healthy and EAE WT mice. The percentage of activated TACE does not vary between the genotypes in both conditions (FIG. 2e).

Cortex: In this CNS area pro-TACE expression was up regulated in disease condition in both PTPH1-KO and WT animals (P_{WT}=0.0027; P_{KO}=0.02 (WT vs. KO)), but the extent of increase was significantly lower in KO compared to WT animals (FIG. 2c). Indeed there was a highly significance decrease in KO pro-TACE expression in diseased condition compared to WT one (P<0.0001) (FIG. 2c). As for cortical mature TACE, its expression was increased due to EAE at 50 dpi in PTPH1-WT (P_{WT}=0.00006 (EAE vs. CTRL)) and in lower extent in PTPH1-KO mice (not sign P_{KO}=0.0692 (EAE vs. CTRL)) (FIG. 2d). As for the percentage of activated TACE, PTPH1-KO mice displayed a significantly lower expression in the cortex in control condition compared to EAE WT littermates (P_{ctrl}<0.001 (WT vs. KO)), but no differences were detected 50 dpi in diseased condition (FIG. 2f).

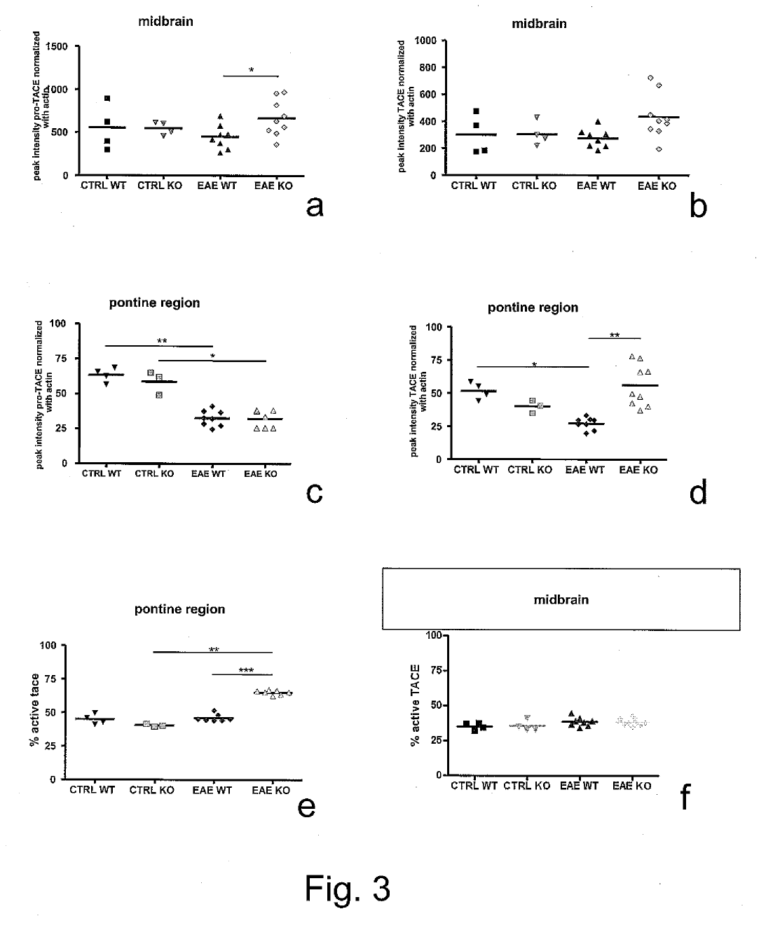

Midbrain: In the midbrain, pro-TACE expression under disease conditions at 50 dpi was slightly higher in PTPH1-KO animals compared to WT littermates (p=0.0261 (WT vs. KO)). This trend of expression was conserved, even though not significantly, in the mature form of TACE (p=0.0645) (FIG. 3a-b). No differences were detected in the percentage of activated TACE over the total amount of TACE proteins (FIG. 3f).

Pontine Region: pro-TACE expression in the pontine region was not significantly different between the genotypes (P_{WT}=0.0024; P_{KO}=0.0154 (EAE vs CTRL)) and the differences recorded were caused by EAE (FIG. 3c). The mature TACE expression decreased in the WT mice due to the disease at 50 dpi, but increased in the EAE KO versus control KO mice. Furthermore, under disease conditions, there was a highly significant increase in mature TACE expression in PTPH1-KO pontine region compared to WT (p=0.001 (WT vs. KO)) (FIG. 3d). The percentage of activated TACE was significantly increased in the PTPH1-KO diseased mice versus the healthy controls (P_{KO}<0.001 (EAE vs CTRL)) and also versus the WT diseased littermates (P_{EAE}<0.001 (WT vs KO)) (FIG. 3e). Taken together, these data corroborate that silencing PTPH1 leads to an increase in TACE expression in the pontine region.

### α-Secretase Activity Test

Proteins extracted from different brain regions were tested for TACE activity by addition of a TACE-specific peptide conjugated to the reporter molecules EDANS and DABCYL. In the uncleaved form the fluorescent emissions from EDANS are quenched by the physical proximity of the DABCYL moiety, which exhibits maximal absorption at the same wavelength (495 nm). Cleavage of the peptide by the α-secretase physically separates the EDANS and DABCYL, leading to the release of a fluorescent signal. The level of TACE enzymatic activity in the cell lysate is hence proportional to the fluorometric reaction.

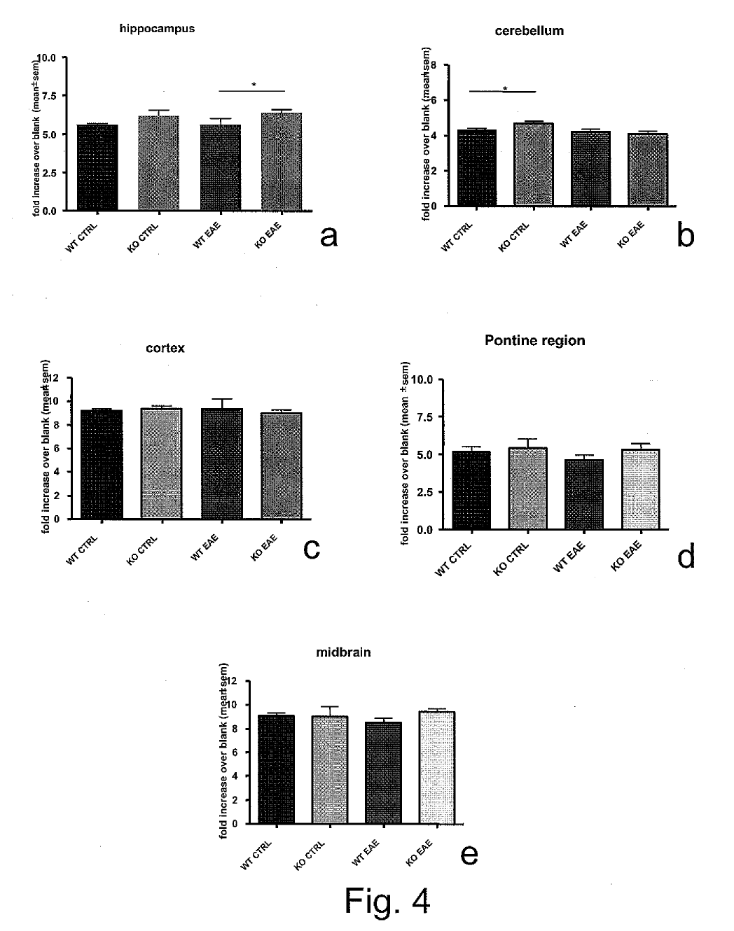

PTPH1-KO EAE mice displayed a slightly higher TACE activity in hippocampus (T-test, p=0.0452), pontine region and midbrain compared to WT diseased littermates (FIG. 4), in agreement with the data on protein expression obtained by WB (Tab. 1). This means that the increased protein activity is due to increased amount of protein, indicating inhibition of TACE expression and activity by PTPH1 under challenged conditions.

TABLE 1
Summary of TACE expression and activity in the different brain
areas of PTPH1-KO versus-WT mice in disease conditions

| | pro-TACE | TACE | TACE activity |
|---|---|---|---|
| CEREBELLUM | ns | ↓ | ns |
| HIPPOCAMPUS | ns | ↑ | ↑ |
| STRIATUM | ns | ns | ns |
| CORTEX | ↓ | ns | ns |
| MIDBRAIN | ↑ | ns | ↑(ns) |
| PONTINE REGION | ns | ↑ | ↑(ns) |

### Conclusions

PTPH1 involvement in AD pathology has been investigated considering its interaction with TACE. TACE, an α-secretase, is localized in the pyramidal neurons of the neocortex, in the granular neurons of the hippocampus and in the Purkinje neurons of the cerebellum (Skovronsky et al., 2001). Skovronsky and colleagues also found that TACE-expressing neurons were often co-localized with AD senile plaques, and in some case were surrounded by them in the hippocampus and cortex.

A preliminary experiment had been carried out to investigate TACE expression and activity in basal conditions. TACE expression and activity have been recorded in PTPH1-KO and WT mice at different brain areas (olfactory bulbs, cerebellum, hippocampus, striatum, cortex, pontine region and midbrain) and no significant differences were recorded (data not shown).

This could be due to some compensatory events occurring in vivo. Therefore, a challenge on the PTPH1-KO mice was the next step tested.

It was decided to move to a model characterized by diffuse CNS inflammation, in which TACE plays a pivotal role, namely the mouse chronic EAE model.

We investigated TACE expression in the brains of late stage mouse chronic EAE (50 days post-immunization) induced by MOG-peptide in PTPH1-KO and WT mice, in order to assess a difference in the extent of central inflammation in upper CNS linked to the genotype. We did not investigate TACE expression in the spinal cord since PTPH1 is not expressed in this CNS area.

Hippocampus, midbrain (thalamic nuclei) and pontine region of PTPH1-KO displayed increased level of TACE expression and activity compared to their WT littermates (Table 1), corroborating that PTPH1 inhibits TACE in vivo. Mouse chronic EAE is an ascending paralysis, induced in the hind limbs and running through the spinal cord. The pontine region, cerebellum and midbrain are the first upper CNS area connected to the spinal cord, and therefore an increased inflammatory process was first expected in theses brain areas. In those areas, which are particularly involved in AD, a difference in TACE expression was indeed noticed between the two genotypes.

It is worth considering that the peak of inflammation in this EAE model is at 15 dpi. After that, the inflammatory process starts to decrease and neurodegeneration becomes the major pathological event leading the disease. The increased TACE expression/activity in PTPH1-KO pons and midbrain seems to reflect an inflammatory response still ongoing in these mice, while it is decreased in the PTPH1-WT littermates. The decrease in TACE level in cortex and cerebellum could be explained by some compensatory activities or a dilution effect.

In summary the above-presented data showed that silencing PTPH1 does not modulate

TACE expression and activity under normal, basal conditions (in CFA immunized mice). On the other hand these data (presented above) on the mouse chronic EAE showed that PTPH1 silencing affects TACE expression and activity in the hippocampus and in the thalamic nuclei (midbrain). A slight modulation was detected also in the cortex. PTPH1-KO EAE mice displayed lower level of TACE activity and expression in the hippocampus and midbrain as compared to PTPH1-WT EAE littermates.

We have thus demonstrated that:
PTPH1 is localized in brain areas affected by Alzheimer's Disease pathology (in a mouse model of CNS inflammation); and
PTPH1 is an in vivo TACE inhibitor in those brain areas, which are strongly involved in Alzheimer's Disease pathogenesis and progression.

This is the first study focused on the role of PTPH1 in CNS diseases and inflammation. It is furthermore the first proof of an in vivo action of this phosphatase on TACE expression and activity in the mouse chronic EAE model.

The conclusion from this study is that PTPH1-inhibitors can be useful in Alzheimer's Disease pathogenesis, lowering the amount of APP that can be converted into Aβ peptide. It is also worth considering that TACE acts as protease of pro-inflammatory cytokines and cytokine receptors, enhancing the inflammatory aspect of the disease, representing a possible unwanted side effect.

### Example 2

### Effect of PTPH1 Silencing on Full Length Aβ Production In Vitro

In this experiment, APP stably transfected cell lines are being used (HEK293-APP_{swedish} and/or CHO-APP_{swedish}) (Qin et al., 2003; Qin et al., 2006; Feng et al., 2006). These cells express the human form of APP with the double Swedish mutation (Lys670→Asn and Met671→Leu), which results in the over-production of the full length Aβ in the medium. PTPH1 siRNA is tested on HEK293-APP_{swedish} and/or CHO-APP_{swedish} cells to further elucidate the role PTPH1 in the pathophysiology of Alzheimer's Disease.

### Cell Culture and Treatment

CHO_{swedish} cells are maintained in MEMα+5% fetal bovine serum (FBS) with added penicillin/streptomycin and glutamine. The cells are transfected with/without PTPH1 siRNA or controls as follows.

Qiagen Mouse ppase library set V1.0 plate A

| | |
|---|---|
| AACCTTTAAAGTTAACAAACAA | (SEQ ID NO: 3) |

Qiagen Mouse ppase library set V1.0 plate B

| | |
|---|---|
| CAGGAGCAAACCAGGCATCTA | (SEQ ID NO: 4) |

As negative control, either antisense sequences of these oligonucleotides or oligonucleotides encoding scrambled sequences of these peptides, are used.

### RT-PCR

RNA is extracted from the cell cultures and analyzed to check the expression of TACE and PTPH1.

The thermal cycling parameters used to perform the RT-PCR assays has been: 50° C. for 2 minutes, 95° C. for 10 minutes, and then 50 cycles of melting at 95° C. for 15 seconds and annealing/extension at 60° C. for 1 minute. TACE (ADAM17) primers are designed by Applied Biosystem # Mm00456428_m1 for mouse and Hs01041927_m1 for human.sense primer (5_-GACTCTAGGGTTCTAGCCCA-3_) (SEQ ID NO: 6) and the TACE antisense primer (5_-CCTCTGCCCATGTATCTGTA-3_) (SEQ ID NO: 7) (Franchimont et al 2005). PTPH1 (PTPN3) primer for mouse are custom-made and the sequences are: forward CGT GTC CCG AGA AAT GCT AGT TA (SEQ ID NO: 8) and reverse: GAG ATG GGT CAC TGT GTG TTC TTC (SEQ ID NO: 9).

### PTPH1 Activity

PTPH1 activity is assessed on cell homogenate using a 6,8-difluoro-4-methylumbelliferyl phosphate (DiFMUP) as a substrate in a test as described below in Example 3.

### ELISA for Aβ40

Protein extract and supernatants from treated and untreated cells are analyzed by Elisa for Aβ40 (Beta-Amyloid 1-40 ELISA Kit, SIGNET, #SIG-38940) in order to confirm that PTPH1 silencing has affected APP processing, a lower amount of Aβ40 indicating PTPH1 inhibition.

### TACE Activity

The protein extract is also tested for TACE activity by using a fluorometric kit of R&D Systems (α-secretase activity kit #FP001). The level of α-secretase enzymatic activity in the cell lysate is proportional to the fluorometric reaction. The analysis is run in duplicates and the results are expressed as fold increases in fluorescence over background controls (reactions without cell lysate or substrate).

### CBA (Cytometric Bead Array)

Cytokines profile for inflammation is assessed on cell medium by human CBA kit (BD Pharmingen) to analyze the direct or indirect involvement of PTPH1 in cytokine release modulation.

### NO Production

One hundred microliters of cell medium are collected and assayed for NO levels with the Griess Reagent (Mol Probes, G-7921) (Green et al., 1982). The release of NO is determined indirectly by measuring the absorbance at 540 nm. Duplicate measurements are obtained for each sample. The remainder of each sample is used for an MTT assay (cell proliferation/growth assay) to normalize the Griess values for cell viability and number. MTT solution (3-(4,5-Dimethylthiazol-2-yl)-2,5-diphenyltetrazolium bromide, 1:1000 dilution) is mixed with the sample and then incubated for 2 h at 37° C., 5% CO₂. After incubation, the cell medium is removed, and cells lysed by the addition of 500 μl of DMSO and rocking at room temperature for 10 min in the dark. Two hundred microliters of lysate are transferred to a 96-well plate, and the absorbance at 550 nm measured.

NO production is a tool to measure oxidative stress in these cells. Oxidative stress is known to contribute to tissue damage during inflammation in general and in the pathogenesis of AD in particular (Law et al., 2001; Yao et al., 2004; Green P. S., et al. 2004). It is thus interesting to understand the role of PTPH1 in this specific aspect of AD inflammation.

### ROS Production

Superoxide anion produced during the respiratory burst can be evaluated using the reduction of nitroblue tetrazolium (NBT) assay (SIGMA). Aliquots of 250 μl of CHO-APP_{swedish} cells (10×10⁶/ml) are mixed with 250 μl of NBT (1 mg/ml) in Hank's balanced salt solution (HBSS) (Invitrogen-GIBCO) and incubated for 30 min at 37° C., the reaction is stopped with 0.5 M HCl, and cells are centrifuged. Supernatants were discarded, and the reduced NBT was extracted with dioxan. Supernatant absorbance at 525 nm was determined in a spectrophotometer. Experiments are performed in duplicate.

ROS production is a tool to measure oxidative stress in these cells. Oxidative stress is known to contribute to tissue damage during inflammation in general and in the pathogenesis of AD in particular (Law et al., 2001; Yao et al., 2004; Green P. S., et al. 2004). It is thus interesting to understand the role of PTPH1 in this specific aspect of AD inflammation.

A cellular assay as described above can be carried out on human cells, transfected with human APP with the double Swedish mutation (HEK293-APP_{swedish} see above). In this case, human siRNA sequences (specific for the human PTPH1 gene) are being used, available e.g. from Ambion. The antisense sequences are being used as negative controls.

| | Sense | antisense |
|---|---|---|
| Ambion | CCAAAAAGUCGGUAAAUAAtt | UUAUUUACCGACUUUUUGGtg |
| 114278 | SEQ ID NO: 1 | SEQ ID NO: 10 |
| Ambion | GCAGUUAAAAGGAGGUUUCtt | GAAACCUCCUUUUAACUGCtt |
| 114277 | SEQ ID NO: 2 | SEQ ID NO: 11 |

A cellular assay as described above can also be carried out to test candidate chemical compounds, which inhibit PTPH1. In this case, the APP CHO-APP_{swedish} cells are being incubated with a PTPH1 inhibitor or vehicle and the effects, in particular the amount of Aβ40 in the cell extract, are measured as outlined above.

### Example 3

### Test for Measuring the Enzymatic Activity of PTPH1 In Vitro (“DiFMUP” Assay)

The DiFMUP assay allows following the dephosphorylation of DiFMUP (6,8-DiFluoro-4-MethylUmbelliferyl Phosphate), which is a PTPH1 substrate, mediated by PTPH1 into its stable hydrolysis product, i.e. DiFMU (6,8-difluoro-7-hydroxy coumarin). Due to its rather low pKa and its high quantum yield, DiFMU allows measuring both acidic and alkaline phosphatase activities with a great sensitivity.

Five μl of diluted candidate compound or vehicle (100% DMSO) are distributed to a 96 well plate. 55 μl of DiFMUP (6,8-difluoro-4-methylumbelliferyl phosphate) 5.45 μM diluted in PTPH1 buffer (20 mM Bis Tris HCl pH 7.5, 0.01% Igepal, 1 mM DL-Dithiothreitol) are added, followed by 40 μl of recombinant human PTPH1 enzyme (25 ng/ml) diluted in PTPH1 buffer in order to start the reaction.

After 40 minutes incubation at room temperature, fluorescence intensity is measured on a spectrofluorimeter (excitation at 355 nm, emission at 460 nm). The difference in fluorescence between the sample containing the candidate compound and the sample containing the vehicle accounts for the effect of the candidate compound on PTPH1 activity and thus allows identifying PTPH1 inhibitors or activators.

### REFERENCES

1. Boxer A. L., Rankin K. P., Miller B. L. et al., (2003) Arch Neurol., 60:949-56
2. Chetelat G. and Baron J C, (2003) Neuroimage, 18: 525-541
3. Comabella M., Romera C., Camina M., Perkal H. et al., (2006) J. Neurol, 253: 701-706
4. Citron M., (2004), Nat. Rev. Neurosci., 5: 677-685
5. Corder, E. H., Saunders, A. M., Strittmatter, W. J., et al.; (1993) Science 261: 921-923,
6. Devanand D. P., Pradhaban G., Liu X. et al., (2007) Neurology, 68: 828-836
7. Feng X., Zhao P., He Y. and Zuo Z, (2006) Gene 371, 1: 68-74
8. Frisoni G. B., Testa C., Zorzan A., et al., (2002) J. Neurol. Neurosurg. Psychiatry, 73: 657-664
9. Fox N. C. Warrington E. K., Freeborough P. A., et al., (1996) Brain, 119: 2001-7
10. Green L. C., Wagner D. A., Glogowski J. et al., (1982) Anal. Biochem. 126, 131-138,
11. Green P. S., Mendez A. J., Jacob J S., Crowley J R., et al., (2004) J. Neurochemistry, 90:
12. Huang Y., Xiao Qin Liu, Wyss-Coray T. et al., (2001) Proc Natl Acad Sci USA. 17; 98(15): 8838-8843;
13. Harris F. M, Brecht W. J., Qin Xu, Tesseur I. et al., (2003) Proc Natl Acad Sci USA. 16; 100(19): 10966-10971;
14. Jack C R Jr., Schiung M. M., Gunter J. L., et al., (2004) Neurology, 62: 591-600
15. Karas G. B., Scheltens P., Visser P. J., et al., (2004) Neuroimage, 23: 708-716
16. Law A., Gauthier S, and Quirion R. (2001) Brain Res. Rev. 35, 1: 73-96;
17. Mahley, R. W. & Rall, S. C., Jr. (2000) Annu. Rev. Genomics Hum. Genet. 1: 507-537;
18. Partrige, Adv Drug Deliv Rev. 2007 Mar. 30; 59(2-3):141-52.
19. Plumb J., Cross A. K., Surr J., Haddock G. et al., J Neuroimmunol. 2005 July; 164(1-2):1-9.
20. Qin W., Ho L., Pompl P. N., Peng Y., Zhao Z., et al., (2003) JBC 278, 51: 50970-50977, 2003;
21. Qin W., Yang T., Ho L., Zhao Z., et al., (2006) JBC 281, 31: 21745-21754,
22. Sahin M., Slaugenhaupt S. S., Gusella J. F., Hockfield S. (1995) Proc. Natl. Acad. Sci. USA 92: 7859-7863
23. Sato et al. (2007) Journal of Controlled Release 122 (2007) 209-216
24. Scarmeas et al., NeuroImage 23, 2004, 35-45
25. Schindowski K., Bretteville A., Leroy K., Bégard S., et al., (2006) Am J Pathol. 169(2): 599-616
26. Seifert T., Kieseier B C, Ropele S, Strasser-Fuchs S et al., (2002) Multiple Sclerosis; 8: 447-451
27. Selkoe D. J. (2001) Physiological Review, 81:741-766
28. Sheng et al., Journal of Neuroscience Volume 22, Issue 22, 15 Nov. 2002, 9794-9799
29. Skovronsky D. M., Fath S., Lee V. M., et al., (2001) J. Neurobiol., 49: 40-46
30. Soutschek et al. (2004) Nature 432, 173-178
31. Thomson and Toga, Alzheimer's Disease: MRI Imaging of Progressive Brain Change, The New Encyclopedia of Neuroscience, Online publication 2008
32. Toft-Hansen H., Nuttal R. K., Edwards D. R. and Owens T. (2004) J. Immunol. 173: 5209-5218
33. Whitwell J. L., Przybelsky S. A., Weigand S. D., et al., (2007) Brain, 130: 1777-86
34. Yao Y., Chinnici C., Tang H., Trojanowski J Q et al., (2004) J. Neuroinflamm. 1:21
35. Zheng Y., Schlondorff J. and Blobel C. B. (2002) JBC 277, 45: 42463-42470

1-11. (canceled)

12. A method of treating Alzheimer's Disease, or a symptom thereof comprising the administration of a Protein-tyrosine phosphatase H1 (PTPH1) inhibitor to a subject having Alzheimer's disease or a symptom thereof.

13. The method according to claim 12, wherein the subject has early stage Alzheimer's Disease.

14. The method according to claim 13, wherein said early stage Alzheimer's Disease is transentorhinal stage I-II.

15. The method according to claim 12, wherein said symptom of Alzheimer's disease is selected from the group consisting of the presence of neurofibrillary tangles, amyloid beta depositions, neuronal degeneration, and brain atrophy.

16. The method according to claim 12, wherein said symptom of Alzheimer's disease is selected from the group consisting of dementia, impairment of memory, personality changes, apathy, agitation, irritability, confusion, disorientation, depression, hallucinations, anxiety, and sleep disorders.

17. The method according to claim 12, wherein said inhibitor decreases PTPH1 enzymatic activity.

18. The method according to claim 12, wherein said inhibitor decreases PTPH1 expression.

19. The method according to claim 12, wherein said inhibitor is a siRNA.

20. The method according to claim 12, wherein said inhibitor is formulated for administration in combination with a drug selected from a cholinesterase inhibitor or a glutamate antagonist.

21. A method of identifying a compound useful in treating or preventing Alzheimer's Disease, or a symptom thereof, comprising:
contacting PTPH1 in the presence or absence of a candidate compound in vitro; and
comparing the activity of PTPH1 in the presence of the candidate compound to the activity of PTPH1 in the absence of the candidate compound,
wherein a compound decreasing the activity of PTPH1 is identified as a compound useful in preventing or treating Alzheimer's Disease, or a symptom thereof.

22. The method according to claim 21, wherein the candidate compound is tested on a cell stably expressing amyloid precursor protein, wherein the activity of PTPH1 is being assessed by measuring the amount of amyloid β peptides in the cell extract or supernatant, and wherein a lower amount of amyloid β peptides in presence of the candidate compound as compared to the absence of the candidate compound is indicative of the utility of the candidate compound in treating Alzheimer's Disease, or a symptom thereof.
